(19) United States
(12) Patent Application Publication
(10) Pub. No.: US 2015/0011398 A1
(43) Pub. Date: Jan. 8, 2015

(54) METHODS FOR QUANTITATIVE DETERMINATION OF PROTEIN-NUCLEIC ACID INTERACTIONS IN COMPLEX MIXTURES

(71) Applicants: Kim Lewis, Newton, MA (US); Michael Sherman, Newton, MA (US)

(72) Inventors: Kim Lewis, Newton, MA (US); Michael Sherman, Newton, MA (US)

(21) Appl. No.: 14/306,580

(22) Filed: Jun. 17, 2014

Related U.S. Application Data
(60) Provisional application No. 61/835,829, filed on Jun. 17, 2013.

(51) Int. Cl. C12N 15/10

(52) U.S. Cl. CPC C12N 15/1068; C12N 15/1037
USPC 506/2

(57) ABSTRACT

In various embodiments, the present invention relates generally to analysis of complex mixtures and, more specifically, to detection and quantitative determination of multiple proteins, protein modifications, and protein-nucleic acid interactions in those complex mixtures.

### CROSS-REFERENCE TO RELATED APPLICATIONS

This application claims priority to and the benefit of, and incorporates herein by reference in its entirety, U.S. Provisional Patent Application No. 61/835,829, entitled QUANTITATIVE DETERMINATION OF MULTIPLE PROTEINS, PROTEIN-PROTEIN AND PROTEIN-NUCLEIC ACID INTERACTIONS IN COMPLEX MIXTURES, which was filed on Jun. 17, 2013.

### FIELD OF THE INVENTION

In various embodiments, the present invention relates generally to analysis of complex mixtures and, more specifically, to detection and quantitative determination of multiple proteins, protein modifications, and protein-nucleic acid interactions in those complex mixtures.

### BACKGROUND

Today, two major approaches are commonly used to evaluate the multiple proteins in complex mixtures: mass spectroscopy and immunological detection with multiplexing. Mass spectroscopy is not readily scalable, and requires the use of radioactive labels to provide quantitative results, which is not compatible with some cell lines. Immunological detection typically uses labeling antibodies with multiple colors, which tends to limit the number of detected proteins to several dozen. Both approaches are also relatively expensive. A need therefore exists for an easily scalable method for quantitatively detecting and determining dozens, hundreds, or thousands of unique proteins in a complex mixture.

### SUMMARY

Embodiments of the present invention allow for detection and quantitative determination of multiple proteins, protein modifications, and protein-nucleic acid interactions.

In some embodiments, the invention pertains to use of an identification vehicle having a binding portion and a nucleic acid portion, where the binding portion is specific for a nucleic acid-binding protein, and where the nucleic acid portion ligates to a nucleic acid fragment bound by the nucleic acid-binding protein, thus forming a complex including the identification vehicle, the nucleic-acid binding protein, and the bound nucleic acid fragment. The bound nucleic acid fragment and nucleic acid portion are isolated and sequenced to identify the binding portion, which in turn identifies the nucleic acid-binding protein that the binding portion is specific for, and also identify a nucleic acid sequence in proximity to where the protein binds the nucleic acid. By adding a known amount of each identification vehicle to a complex mixture, and then identifying the identification vehicles, the relative and/or absolute amounts of each protein in the mixture can be quantified.

In further embodiments, the invention pertains to use of an identification vehicle having a binding portion and a nucleic acid portion, where the nucleic acid portion encodes the binding portion. Using a display library provides identification vehicles displaying a binding portion specific for a protein target of interest, such a fusion viral coat protein displaying an antibody, while the nucleic acid portion, such as the phage genome in a phage display library, encodes the binding portion. By adding a known amount of each identification vehicle to a complex mixture, and then identifying the identification vehicles (by sequencing nucleic acids), the relative and/or absolute amounts of each target of interest in that mixture can be quantified.

Accordingly, in one aspect, the invention pertains to a method of detecting and quantifying a plurality of unique proteins present in a mixture, the method comprising the steps of providing a plurality of identification vehicles each consisting essentially of (a) a binding portion specific to a unique protein in the mixture and (b) a nucleic acid portion encoding the binding portion; incubating the identification vehicles with the mixture, whereby the binding portions of at least some of the vehicles bind to corresponding proteins in the mixture; collecting bound identification vehicles; and sequencing at least a portion of the associated nucleic acids to quantify the proteins corresponding thereto. In some embodiments, the identification vehicles are derived from a display library. In further embodiments, the display library is at least one of a CIS display library, a dsDNA display library, an IVC display library, a cell surface display library, a ribosome display library, or an mRNA display library. In certain embodiments, the display library is a phage display library. In further embodiments, the binding portion is one of an antibody, Fab antibody fragment, F(ab′)₂ antibody fragment, Fab′ antibody fragment, single-chain variable fragment, dimeric single-chain variable fragment, single domain antibody fragment, bi-specific antibody, heavy-chain antibody, oligonucleic acid aptamer, peptide aptamer, virus, or peptide-bound virus. In some embodiments, the proteins in the mixture are immobilized on a solid support. In further certain embodiments, the binding portion is specific to a modified protein. In certain embodiments, the modified protein is modified by at least one of phosphorylation, acetylation, glycosylation, ubiquitination, SUMOylation, methylation, and glutathionation. In further embodiments, the step of sequencing at least a portion of the associated nucleic acids includes sequencing the nucleic acid portions of at least two identification vehicles, each identification vehicle including a binding portion specific to a different protein, and comparing the number of sequence reads to quantify the relative frequency of the proteins targeted by the binding portions associated with the sequenced nucleic acid portions. In some embodiments, the method further comprises the step of removing from the mixture unbound identification vehicles subsequent to the step of incubating the identification vehicles with the mixture.

In another aspect, the invention pertains to a method of analysis of mechanism of drug action, the method comprising the steps of acquiring a proteome of an organism prior to administration of a drug to the organism; acquiring the proteome of the organism subsequent to administration of the drug to the organism; comparing the proteomes acquired prior and subsequent to administration of the drug using the method of detecting and quantifying a plurality of unique proteins present in a mixture described herein.

In a further aspect, the invention pertains to a method of detecting and quantifying a plurality of unique proteins present in a mixture, at least some of the unique proteins binding in the mixture to nucleic acid fragments, the method comprising the steps of providing a plurality of identification vehicles each consisting essentially of (a) a binding portion specific to a unique protein in the mixture, and (b) an oligonucleotide portion; incubating the identification vehicles with the mixture, whereby the binding portions of at least some of the vehicles bind to corresponding proteins in the mixture; ligating the oligonucleotide portions of bound identification vehicles with the nucleic acid fragments bound to proteins to which the binding portions of the identification vehicles are themselves bound, thereby forming complexes each having a protein, a binding vehicle, and a combined nucleic acid strand including the oligonucleotide of the identification vehicle and a protein-bound nucleic acid fragment; and sequencing at least a portion of the combined nucleic acid strands to quantify the proteins corresponding thereto and identify the nucleic acid fragments to which the proteins bind. In some embodiments, the nucleic acid fragment is a DNA fragment. In further embodiments, the nucleic acid fragment is a RNA fragment.

In certain embodiments, at least some of the unique proteins bind in the mixture to RNA fragments and the oligonucleotide portions of the identification vehicles each comprise a terminal sequence complementary to a portion of the RNA, the method further comprising the steps of annealing the terminal sequences of bound identification vehicles with complementary portions of the RNA fragments bound to proteins to which the binding portions of the identification vehicles are themselves bound, thereby forming complexes each having a protein, a binding portion, and a combined nucleic acid strand including the oligonucleotide portion of the identification vehicle and a protein-bound RNA fragment; and extending the oligonucleotides along the RNA fragments to which they are bound to form, from each oligonucleotide, a DNA strand including the oligonucleotide and a terminal portion complementary to at least a portion of the bound RNA fragment; and isolating the DNA strands, wherein the determining step identifies the protein and a sequence of at least a portion of the DNA fragment. In some embodiments, two or more different identification vehicles against the same proteins in the mixture are present in the incubation reaction. In further embodiments, the oligonucleotide is a single strand DNA, double strand DNA, triple strand DNA, or quadruple strand DNA, or single strand RNA, or double strand RNA, triple strand RNA, or quadruple stand RNA molecule. In certain embodiments, the oligonucleotide portion includes a first oligonucleotide strand attached to the binding portion and a second oligonucleotide strand complementary to the first oligonucleotide strand and annealed thereto. In some embodiments, the second oligonucleotide strand includes unblocked 3′- and 5′-ends. In certain embodiments, the step of incubating the identification vehicles with the mixture includes the binding portions of at least some of the vehicles bind to corresponding proteins in the mixture, wherein at least some of the corresponding proteins are bound to the same nucleic acid fragment; and the step of ligating the oligonucleotide portions of bound identification vehicles includes ligating to each other the oligonucleotide portions of identification vehicles bound to corresponding proteins bound to the same nucleic acid fragment, and ligating the oligonucleotide portions to the nucleic acid fragment, thereby forming complexes each having proteins, binding vehicles, and a combined nucleic acid strand including the oligonucleotides of each identification vehicle and a protein-bound nucleic acid fragment.

In some embodiments, the oligonucleotide portion is the binding portion of the identification vehicle. In further embodiments, the oligonucleotide portion is a modified oligonucleotide. In certain embodiments, the modified oligionucleotide is modified by incorporation of at least one of an inhibitor of nuclease degradation, a fluorescent dye, a dark quencher, a locked nucleic acid, an unlocked nucleic acid, a modified base, a modified sugar, a threose nucleic acid, a glycol nucleic acid, a peptide nucleic acid, a zip nucleic acid, a triazole-linked deoxyribonucleic acid, a morpholino synthetic nucleic acid, a spacer, or biotin. In some embodiments, the binding portion is one of an antibody, Fab antibody fragment, F(ab′)₂ antibody fragment, Fab′ antibody fragment, single-chain variable fragment, dimeric single-chain variable fragment, single domain antibody fragment, bi-specific antibody, heavy-chain antibody, oligonucleic acid aptamer, or a peptide aptamer. In further embodiments, method further comprises the step of removing unbound identification vehicles from the mixture subsequent to the step of incubating the identification vehicles with the mixture. In certain embodiments, the method further comprises the step of isolating the combined nucleic acid strands prior to the sequencing step.

In another aspect, the invention pertains to a method for drug quality control, the method comprising the steps of acquiring a proteome of an organism prior to administration of a drug to the organism, acquiring the proteome of the organism subsequent to administration of the drug to the organism, and comparing the proteomes acquired prior and subsequent to administration of the drug using the methods described herein.

In a further aspect, the invention pertains to a method for drug quality control, the method comprising the steps of separating cells or tissues into a first portion and a second portion, incubating the first portion with a drug, a second portion not being incubated with the drug, and comparing the first portion and second portion using the methods described herein.

In another aspect, the invention pertains to a method of analysis of mechanism of drug action, the method comprising the steps of acquiring a proteome of an organism prior to administration of a drug to the organism, acquiring the proteome of the organism subsequent to administration of the drug to the organism, and comparing the proteomes acquired prior and subsequent to administration of the drug using the methods described herein.

In a further aspect, the invention pertains to a method of analyzing a mechanism of drug action, the method comprising the steps of separating cells or tissues into a first portion and a second portion, incubating the first portion with a drug, a second portion not being incubated with the drug, and comparing the first portion and second portion using the methods described herein.

In another aspect, the invention pertains to a method of analyzing the effectiveness of a drug, the method comprising the steps of acquiring a proteome of an organism prior to administration of a drug to the organism, acquiring the proteome of the organism subsequent to administration of the drug to the organism, and comparing the proteomes acquired prior and subsequent to administration of the drug using the methods described herein.

In a further aspect, the invention pertains to a method of analyzing the effectiveness of a drug, the method comprising the steps of separating cells or tissues into a first portion and a second portion, incubating the first portion with a drug, a second portion not being incubated with the drug, and comparing the first portion and second portion using the methods described herein.

In a further aspect, the invention pertains to a method of identifying and validating biomarkers for a trait, the method comprising providing a first sample of cells or tissues positive for the trait and a second sample of cells or tissues negative for the trait, and comparing the first sample and second sample using the methods described herein.

In a certain embodiment, the invention pertains to a diagnostic method for identifying a trait in a biological sample, the method comprising evaluating the sample using the methods described herein.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 is a diagram illustrating an embodiment of a method for quantitative identification of multiple proteins in a sample.

FIG. 2A is a graph illustrating FACS analysis of expression of receptor A in low expression cells.

FIG. 2B is a graph illustrating FACS analysis of expression of receptor A in high expression cells.

FIG. 3 is a diagram illustrating interactions between phage antibodies and antigens on receptor A.

FIG. 4A is a graph comparing relative expression of receptor A detected using three different phages from the phage display method described in Example 3 and detected using conventional FACS analysis.

FIG. 4B is a graph comparing relative expression of receptor A detected using an average of the phage data from FIG. 8A (referred to here as Proteome™) and detected using conventional FACS analysis.

FIG. 5 is a diagram illustrating an embodiment of a method for quantitative identification of proteins interacting with DNA in a sample.

FIG. 6A is an illustration of an embodiment of an antibody with attached oligonucleotide and complementary oligonucleotide.

FIG. 6B is an illustration of an embodiment of a method for quantitative identification of interaction between multiple proteins and DNA in a sample.

FIG. 7A is an image of an electrophoresis gel indicating antibody-oligonucleotide conjugation.

FIG. 7B is an image of an electrophoresis gel indicating the presence of HSF1 binding sites.

FIG. 7C is an image of an electrophoresis gel indicating amplification of genes associated with HSF1.

FIG. 7D is an image of an electrophoresis gel indicating binding between HSF1-Ab-oligo conjugates and DNA in proximity to known HSF1 binding sites.

FIG. 8 is a diagram illustrating an embodiment of a method for quantitative identification of proteins interacting with RNA in a sample.

### DETAILED DESCRIPTION

Embodiments of the present invention facilitate quantitative detection of multiple species in a complex mixture. In various implementations, identification vehicles are formed, each including a binding portion and nucleic acid portion associated with the binding portion. The binding portion is specific for a target of interest. For example, in some embodiments, the binding portion is an antibody specific for a nucleic-acid binding protein, and the nucleic acid portion encodes the antibody. In further embodiments, the binding portion is specific to a protein modified by post-translational modification, such as, for example, phosphorylation, acetylation, glycosylation, ubiquitination, SUMOylation, methylation, and glutathionation. In further embodiments, the binding portion is one of an antibody, Fab antibody fragment, F(ab′)2 antibody fragment, Fab′ antibody fragment, single-chain variable fragment, dimeric single-chain variable fragment, single domain antibody fragment, bi-specific antibody, heavy-chain antibody, oligonucleic acid aptamer, peptide aptamer, virus, or peptide-bound virus. Although the ensuing discussion focuses on the use of antibodies as binding molecules, it is to be understood that this is solely for ease of presentation, and that any suitable binding molecule may be used and is within the scope of the invention.

Multiple distinct antibody or antibody fragment binding portions specific to target proteins of interest are linked to nucleotides which encode the linked antibody or antibody fragment. The identification vehicle may be generated, for example, from a phage display library, a CIS display library, a dsDNA display library, an IVC display library, a cell surface display library (generated from, for example, mammalian, yeast, or bacterial cells), a ribosome display library, or an mRNA display library. Using nucleic acid portions encoding the binding portions, as opposed to arbitrary or random nucleic acid sequences, simplifies maintenance of the binding molecule libraries, and allows expansion of the range of the antibodies or other binding molecules to probe a very large number of proteins up to the full proteome.

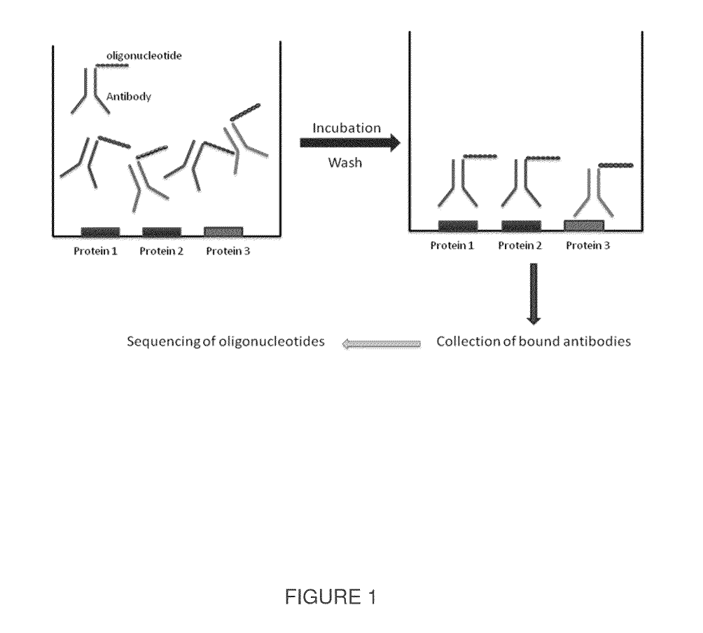

A mixture of identification vehicles is incubated with a complex protein mixture, for example, cell lysate or a proteome, having multiple proteins of interest; for example, the mixture may contain as few as two or as many as 50,000 or more different proteins. The protein-containing mixture to be studied may be immobilized on a surface, such as glass, plastic, or other surfaces known in the art. One skilled in the art will know how to immobilize a protein-containing mixture on a suitable surface without undue experimentation. Unbound identification vehicles are then washed out, and bound identification vehicles are collected. To detect multiple oligonucleotide sequences within the harvested identification vehicles, quantitative DNA sequencing following PCR amplification may be used (see FIG. 1). Since the variety of DNA sequences is practically unlimited, this approach allows large numbers of distinct proteins in a sample to be detected simultaneously.

### Display Libraries

In some embodiments, detection and quantification of proteins in a complex mixture uses identification vehicles from display libraries. The identification vehicles each include a binding portion specific to a unique protein in the mixture and a nucleic acid portion encoding the binding portion. For example, antibody-carrying phages against specific proteins of interest may be selected from a phage display library, isolated and sequenced. Samples containing complex protein mixtures immobilized on a surface are incubated with the mixture of phages carrying Fab fragments against the proteins of interest. Following the incubation, unbound phages are washed out, and phages bound to the sample due to the interaction of their Fab fragments with antigens in the sample are collected. Phage particles carrying Fab fragments against an antigen in the complex protein mixture bind to the sample proportionally to the amount of this antigen, and therefore the number of collected phage particles of this kind is also proportional to the content of the antigen. After collection of bound phages, their DNA is isolated and sequenced, which gives quantitative determination of the presence of DNA signatures of phages carrying each Fab fragment. Collection of bound phages, isolation, and sequencing of phage DNA is a technique known to those of ordinary skill in the art. Since the number of these DNA signatures is proportional to the content of each antigen of interest, the sequencing data are translated into quantitative evaluation of the proteins in the complex mixture. Since the current capacity of NGS is more than a billion DNA signatures, and is expected to further increase over time, this method allows evaluation of a complex protein mixture containing the full human proteome in several samples in one run. Therefore, the method is easily scalable.

In other embodiments, the display library may be a CIS display library, a dsDNA display library, an IVC display library, a cell surface display library, a ribosome display library, or an mRNA display library. The binding portion of the identification vehicle is preferably one of an antibody, Fab antibody fragment, F(ab′)₂ antibody fragment, Fab′ antibody fragment, single-chain variable fragment, dimeric single-chain variable fragment, single domain antibody fragment, bi-specific antibody, heavy-chain antibody, oligonucleic acid aptamer, peptide aptamer, virus, or peptide-bound virus. In some embodiments, the binding portion is specific to a modified protein, such as, for example, phosphorylation, acetylation, glycosylation, ubiquitination, SUMOylation, methylation, and glutathionation. Other protein modifications are known to those skilled in the art.

### EXAMPLE 1

Proteins are quantitatively identified in a sample using a combination of an antibody display library and sequencing. Expression of a cell surface receptor, a recombinant protein referred to herein as receptor A, was compared in HEK293 cells that have high and low levels of receptor A. Comparison was done in parallel using a standard FACS-based procedure and the method described below.

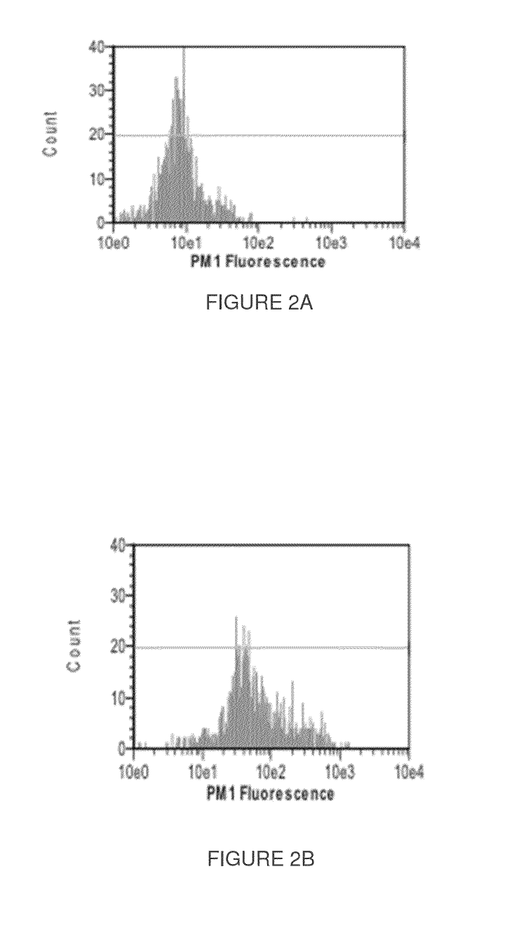

As an initial control experiment, two cell lines with high and low levels of receptor A were grown, harvested, and incubated with commercially available antibodies specific for receptor A for 30 min. Cells were washed 3 times with PBS and incubated with a secondary antibody specific for the first antibody. FACS analysis of these cells lines, as shown in FIGS. 2A and 2B, indicate that the high expression cell lines express receptor A at an approximately tenfold higher amount than the low expression cell lines. More specifically, the FACS analysis provided a mean of 107.9 for the high expression cells and a mean of 11.4 for the low expression cells.

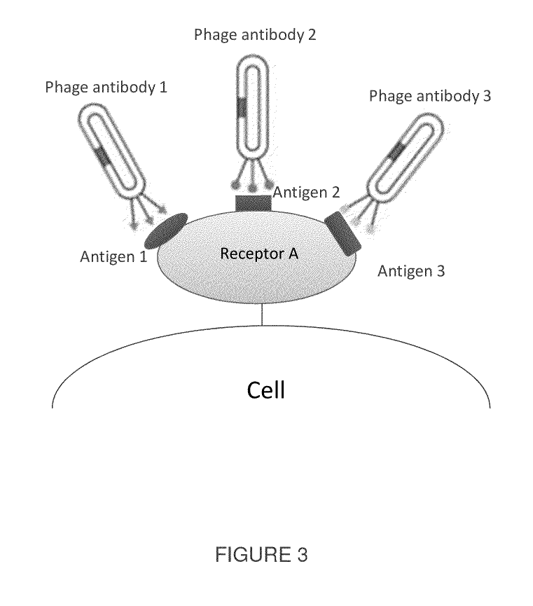

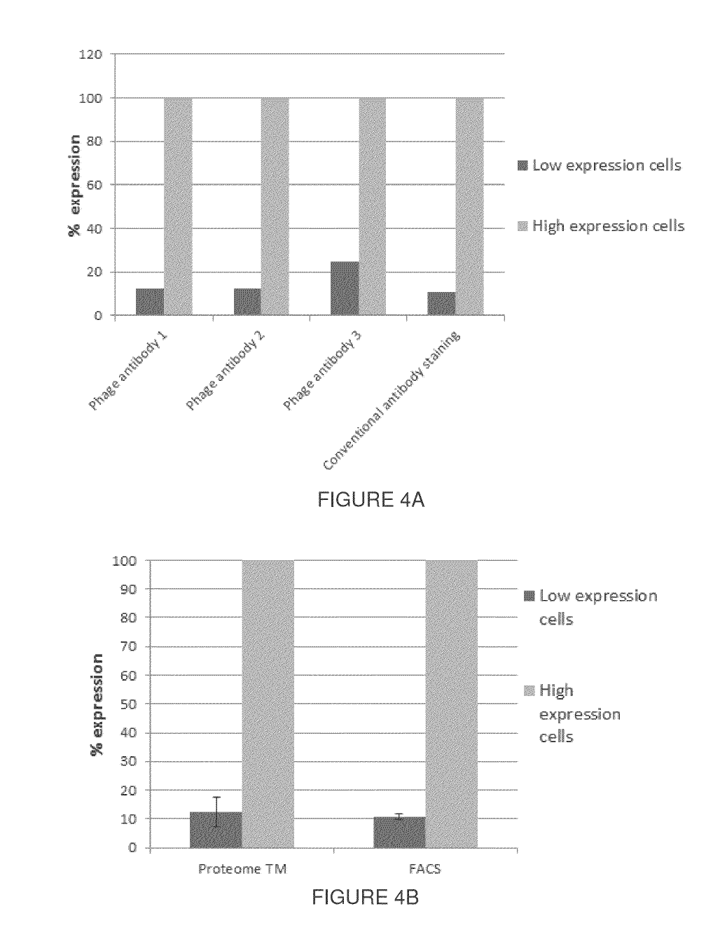

Referring now to FIG. 3, three different M13 phages were prepared, each expressing a Fab fragment (binding moiety) against a different epitope in receptor A fused to its PIII coat protein. Three phages adapted bind to three different epitopes in receptor A were mixed together in equimolar quantities. Cell samples with high and low levels of receptor A expression, the same cell lines as used in the control experiment, were harvested and incubated with the mix of the phages for 30 min. The cells were washed 3 times with PBS. Instead of using commercial antibodies, as in the control experiment, the M13 phages expressing Fab fragments against receptor A were attached to the cells expressing the receptor A. Then the phages were neutralized by incubating at 95° C. for 10 min. DNA from both samples was isolated. Specific DNA sequences were amplified for 10 PCR cycles to prepare the DNA library for the sequencing analysis using Ion Torrent (Life Technologies) NGS technology. Bar codes were attached to both samples (from low and high expression cells) during the PCR amplification step to allow for analysis of all samples in one sequence run. The sequences of the M13 phages were already known. The number of sequence reads of the signatures of Fab sequences indicated the type of phage (one of the three) and the frequency of the receptor A occurrence on the surface of each cell type. Thus low-expression cells had a low number of reads as compared to high-expression cells that contained a high number of reads with all 3 phages used (FIGS. 4A and 4B). As seen in the figures, the analysis indicated similar levels of expression for the receptor A as the standard FACS analysis.

Protein-DNA Interactions

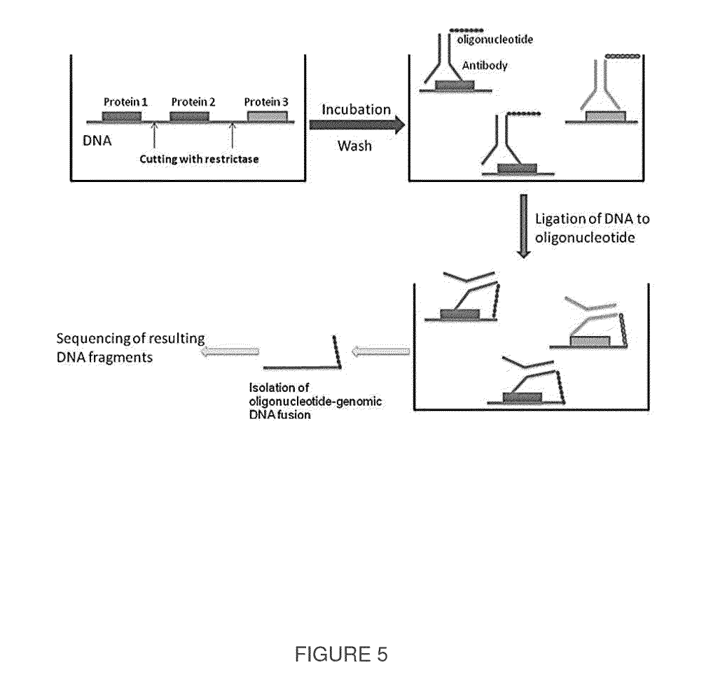

Referring now to FIG. 5, a collection of antibodies against DNA-interacting proteins—such as transcription factors or chromatin modification factors—is used to detect protein-DNA interactions. These antibodies are linked to antibody-specific oligonucleotides (which, again, may have an arbitrary sequence so long as one oligomer is uniquely assigned to a particular antibody type) with a distal restriction site. Following incubation of a sample that contains DNA with proteins bound thereto, the DNA and the antibody-linked oligonucleotides are treated (either separately or, more preferably, together in the mixture) with at least one restriction enzyme, which generates blunt ends or complementary sticky ends on the antibody-linked oligonucleotides and the DNA. In some embodiments, at least one restriction enzyme is selected that generates nucleotide fragments with an average length of approximately 0.5 kB. In other embodiments, at least one restriction enzyme is selected that generates longer or shorter average length nucleotide fragments. In still other embodiments, other means for DNA fragmentation, such as, for example, sonication, can be used to generate DNA fragments with free ligatable blunt ends or sticky ends.

The antibodies bind to the DNA-interacting protein to which each is specific, and the resulting DNA fragments with interacting proteins are ligated to oligonucleotides linked to the antibodies that have bound to these proteins. This ligation generates DNA strands each corresponding to the cleaved DNA portion associated with bound proteins and the oligomer paired therewith via the associated antibody. The generated DNA strands may be isolated from the antibody and binding protein and quantitatively sequenced, such as, for example, by using next generation sequencing (“NGS”) techniques. NGS refers to massively parallel sequential identification of nucleic acid bases in nucleotide sequences as the sequences are re-synthesized from template strands. One skilled in the art will know how to obtain an isolate DNA from the antibody and binding protein and sequence the isolated DNA strand without undue experimentation. The oligonucleotide sequence of a strand identifies the antibody to which it is bound, which in turn identifies the target protein to which the antibody is specific, and the remainder of the strand identifies sites on the genome that are in close proximity to where the target protein binds on the DNA.

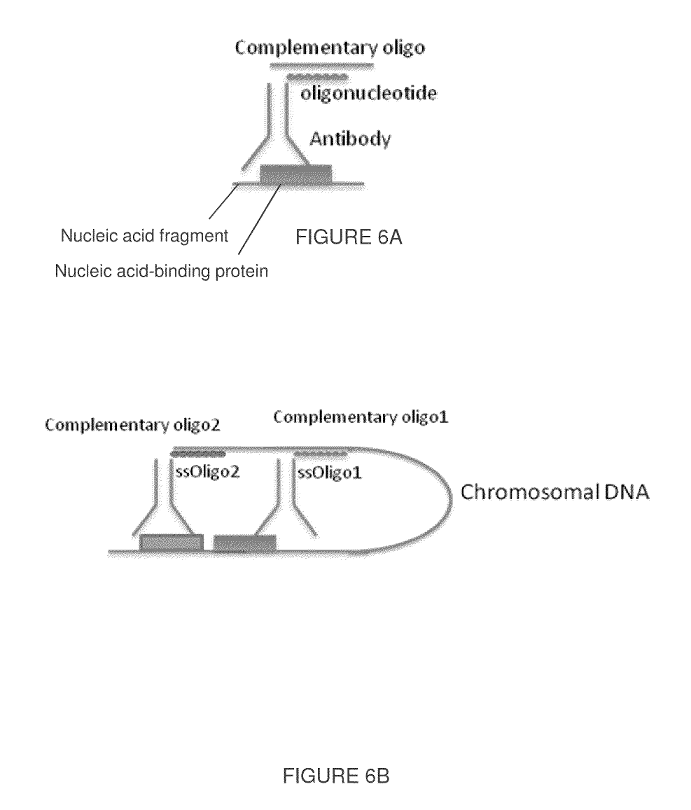

Detecting complexes of two or more proteins associated with DNA can be accomplished by modification of the above-described method that uses ligation of DNA with an identification vehicle consisting essentially of a binding portion, such as an antibody, and an oligonucleotide portion. In some embodiments, the oligonucleotide portion includes a first oligonucleotide strand and a second nucleotide strand complementary to the first strand and annealed thereto, as shown in FIG. 6A. In certain embodiments, the second oligonucleotide strand includes overhangs and/or unblocked 3′- and 5′-ends. With this design, oligonucleotide portions of identification vehicles are capable of ligating both to the DNA bound by proteins targeted by the binding portions and to oligonucleotide portions of other identification vehicles, thus forming complexes having two or more proteins, DNA bound by the proteins, binding portions bound to the proteins, and oligonucleotide portions ligated to each other and to the DNA, as shown in FIG. 6B. Sequencing of this hybrid molecule will identify both the identification vehicles and the DNA. In some embodiments, the DNA is chromosomal DNA.

In some embodiments, the nucleotide portion further includes modifications, such as, for example, an inhibitor of nuclease degradation (for example, a phosphorothioate bond substituting a sulfur atom for a non-bridging oxygen in the phosphate backbone of the oligonucleotide renders the inter-nucleotide linkage resistant to nuclease degradation), a fluorescent dye, a dark quencher, a spacer (for example, hexanediol, triethylene glycol, hexa-thyleneglycol, or 1′,2′-dideoxyribose), biotin, a locked nucleic acid (“LNA”) (a modified ribose backbone with an extra bond connecting the 2′ oxygen and 4′ carbon locks base in the C3′-endo position, increasing Tm and nuclease resistance), an unlocked nucleic acid, a modified base (for example, a 2′-O-methyl RNA base increases a nucleotide's melting temperature (“Tm”) and stability with respect to DNAses and ss ribonucleases), a modified sugar, a threose nucleic acid, a glycol nucleic acid, a peptide nucleic acid, a zip nucleic acid, a morpholino synthetic nucleic acid, or a triazole-linked deoxyribonucleic acid. Numerous modified bases and sugars are known to those skilled in the art.

### EXAMPLE 2

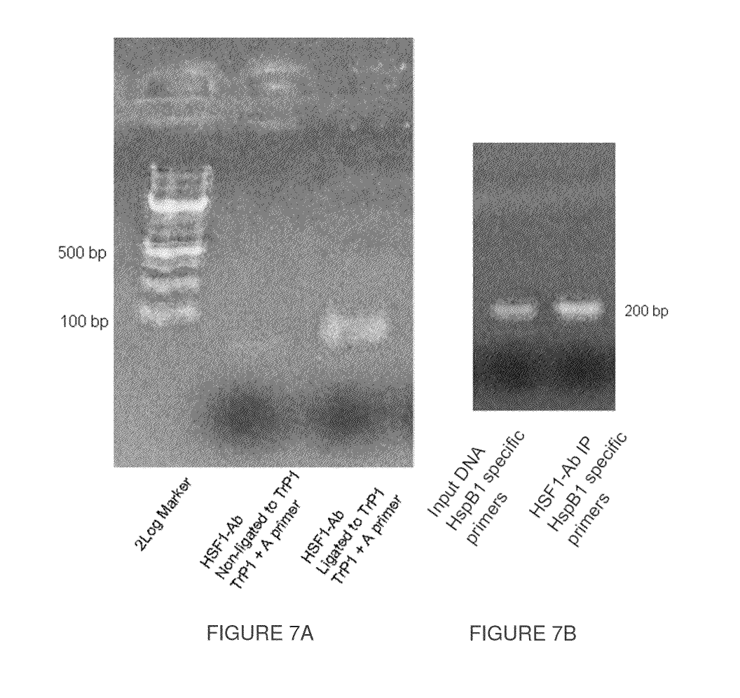

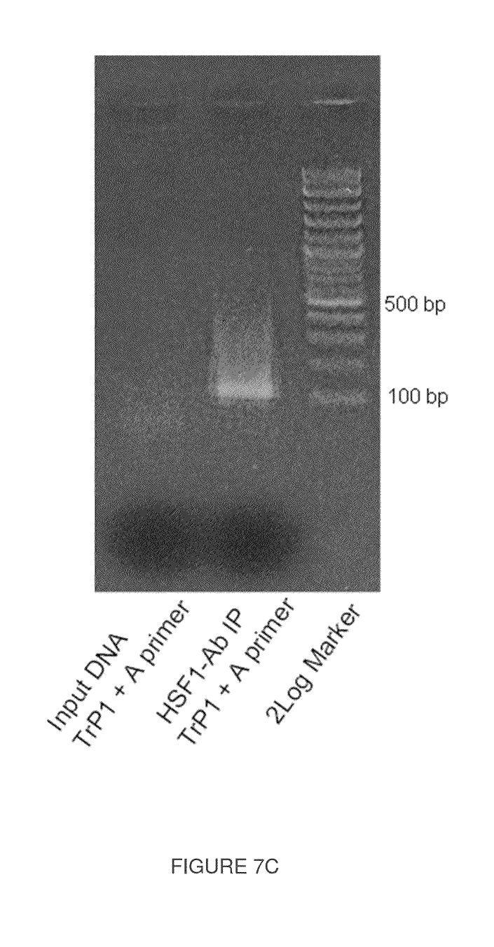

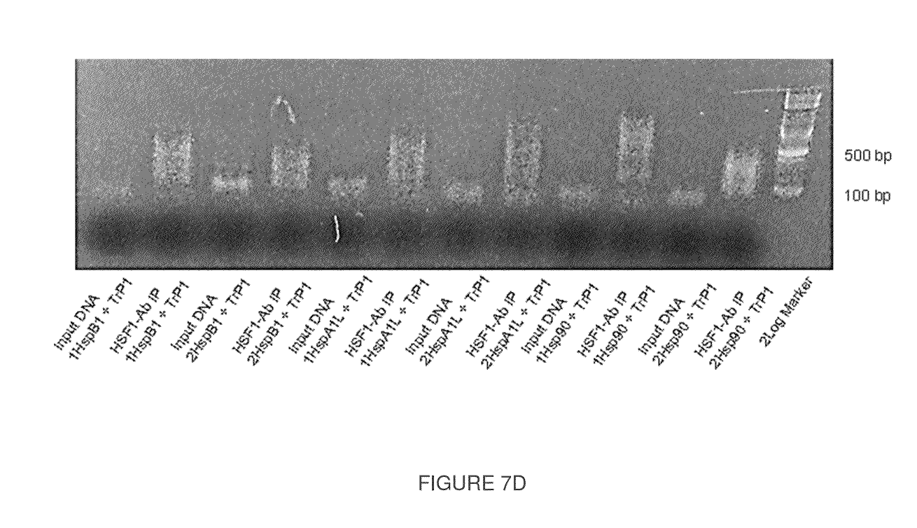

ChIP-grade HSF1 antibody (HSF1-Ab) was conjugated to an oligonucleotide that contained a 5′ adaptor sequence and a signature sequence containing a primer A sequence. To test for the efficiency of conjugation, oligo-HSF1-Ab was ligated to the TrP1 primer. The oligo-HSF1-Ab was purified using Protein A magnetic beads, and the presence of oligonucleotide in the conjugate was confirmed by PCR using A and TrP1, followed by gel electrophoresis (FIG. 7A).

Approximately 5×10⁶ HEK293 cells were harvested. The cells were cross-linked with 1% formaldehyde at room temperature for 10 minutes on a plate rotator, followed by neutralization using 0.2M glycine. The cross-linked chromatin obtained from the fixed cells was subjected to sonication to an average size of 300 bp. The sonicated chromatin was immunoprecipitated using the HSF1 Ab-oligo conjugate. The immunopreciptation was confirmed using specific primers flanking known HSF1 binding sites in HspB1 gene (FIG. 7B).

The chromatin DNA fragments bound to the oligo-HSF1-Ab on Protein A magnetic beads were ligated to the oligos conjugated to the HSF1 antibody using T4 DNA ligase. After one hour, the 3′ adaptor oligo for sequencing (TrP1 oligo) was added to the mixture, and ligation continued.

The sample was treated with proteinase K at 55° C. overnight. The DNA was then isolated. Ligation of the adaptor sequences was confirmed by PCR using primers corresponding to 5′ adaptor sequence attached to HSF1-Ab and the 3′ TrP1 adaptor (FIG. 7C). In this experiment, all Hsf1-associated sequences are amplified by PCR. To test for the specificity of the reactions, PCR reactions were also performed using specific primers flanking known HSF1 binding sites in HspB1, HspA1L and Hsp90 genes, and the results shown via gel electrophoresis (FIG. 7D). These results indicate that HSF1 Ab-oligo conjugate is binding to DNA in proximity to known HSF1 binding sites.

### Protein-RNA Interactions

In some embodiments, detection of protein-RNA interactions uses identification vehicles including binding portions, for example, antibodies against RNA-interacting proteins, and nucleotide portions, such as oligonucleotides. Following incubation of a sample that contains RNA with proteins bound thereto with the identification vehicles, the RNA and the nucleotide portions are ligated (either separately or, more preferably, together in the mixture). This ligation generates RNA strands each corresponding to the RNA associated with bound proteins and the oligonucleotide paired therewith via the associated antibody. These RNA strands may be isolated from the antibody and binding protein, amplified, and used to generate cDNA by a reverse transcriptase, which can be subsequently sequenced. One skilled in the art will know how to obtain a isolate RNA from the antibody and binding protein, prepare cDNA and sequence the isolated cDNA strand without undue experimentation. The oligonucleotide sequence of a strand identifies the antibody to which it is bound, which in turn identifies the target protein to which the antibody is specific, and the remainder of the strand identifies RNA molecules where proteins of interest bind in the sample.

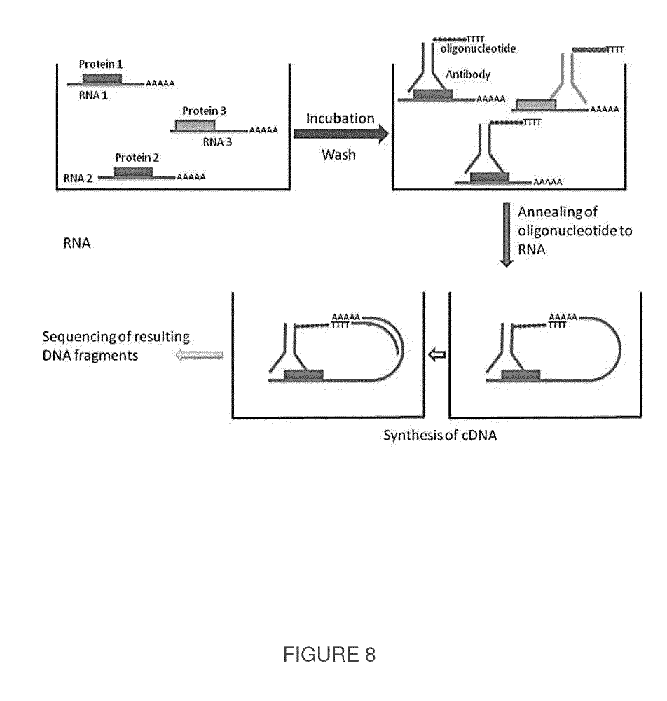

In another approach, as shown in FIG. 8, the oligonucleotides attached to the antibodies include a polyT sequence distal to the antibody-specific sequences. The antibody-linked oligonucleotide preferably binds to the RNA (for example, mRNA) that interacts with the protein to which the antibody binds. The oligonucleotide in the antibody-protein complex is located in close proximity to the RNA, and accordingly, its polyT sequence preferentially binds to the polyA sequence of the mRNA. Following incubation of a sample that contains RNA species bound to proteins with the antibody mixture, the antibody-linked oligonucleotides are allowed to anneal with the polyA sequence on the corresponding mRNAs. The annealed oligonucleotide is then used as a primer to synthesize cDNA on the RNA using a reverse transcriptase. The resulting cDNA strands each contain the original antibody-specific oligonucleotide sequence and a sequence corresponding to the RNA species of the complex. Accordingly, sequencing a DNA strand both identifies the antibody (and, hence, the target protein) and at least a portion of the protein-bound RNA. Typically, the polyT sequence has a length of 5 to 600 nucleotides.

More generally, oligonucleotides useful in the various embodiments described above have a length ranging from 6 to 10,000 nucleotides. The precise length is straightforwardly determined by the skilled practitioner without undue experimentation based on the particular application. For example, longer sequences are more expensive to make, but in embodiments requiring binding to another sequence (as illustrated in FIGS. 3B and 5), a longer binding sequence may result in a greater percentage of bound species. In some embodiments, the oligonucleotide may be a “barcode” nucleotide as described, for example, in Baccaro et al., “Barcoded Nucleotides,” Angewandte Chemie 51(1):254-257 (2012), the entire disclosure of which is hereby incorporated by reference. The optimal length of this sequence for this applications is determined the conditions used for ligation and consequent amplification by PCR. The length of the restriction site depends on enzyme(s) employed, and typically has a length of 4 to 8 nucleotides. In certain embodiments, the nucleotide portion of the identification vehicle further includes modifications, as described above.

### EXAMPLE 3

Antibodies specific for a set of RNA binding proteins are selected. Each antibody is conjugated with its specific signature oligonucleotide (Ab-oligo), such as a barcode or other identifying sequence. Each signature oligonucleotide includes a 5′ adaptor for deep sequencing. The Ab-oligos are mixed together. The HEK293 cells are UV crosslinked. The lysate is prepared according to the CLIP protocol from Ule et al., “CLIP: A method for identifying protein-RNA interaction sites in living cells.” Methods 37 (2005) 376-386. Immunoprecipitation is performed according to CLIP protocol using the Ab-oligo mix as the immunoprecipitating antibodies. Immunoprecipitated RNA is ligated to the Ab-oligo using T4 RNA ligase I (ssDNA from the oligonucleotide is ligated to immunoprecipitated ssRNA). Ligation is continued by adding the 3′ adaptor oligo for sequencing. The sample is treated with Proteinase K at 55° C. overnight. RNA is then isolated from the sample. Nucleic acids are extended using reverse transcriptase (Promega) and Tth DNA Polymerase (Promega) with the adaptor primer. Alternatively, other reverse transcriptases and DNA polymerases can be used. The sample is prepared for sequencing analysis. The sequence of the RNA-signature oligonucleotide-3′ adaptor oligo complex is then determined, identifying the RNA binding protein (via the signature oligo, which identifies the antibody, which identifies the protein for which the antibody is specific) and the RNA sequence where the protein binds.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

1. A method of detecting and quantifying a plurality of unique proteins present in a mixture, at least some of the unique proteins binding in the mixture to nucleic acid fragments, the method comprising the steps of:
providing a plurality of identification vehicles each consisting essentially of (a) a binding portion specific to a unique protein in the mixture, and (b) an oligonucleotide portion;
incubating the identification vehicles with the mixture, whereby the binding portions of at least some of the vehicles bind to corresponding proteins in the mixture;
ligating the oligonucleotide portions of bound identification vehicles with the nucleic acid fragments bound to proteins to which the binding portions of the identification vehicles are themselves bound, thereby forming complexes each having a protein, a binding vehicle, and a combined nucleic acid strand including the oligonucleotide of the identification vehicle and a protein-bound nucleic acid fragment; and
sequencing at least a portion of the combined nucleic acid strands to quantify the proteins corresponding thereto and identify the nucleic acid fragments to which the proteins bind.

2. The method of claim 1, wherein the nucleic acid fragment is a DNA fragment.

3. The method of claim 1, wherein the nucleic acid fragment is a RNA fragment.

4. The method of claim 3, wherein at least some of the unique proteins bind in the mixture to RNA fragments and the oligonucleotide portions of the identification vehicles each comprise a terminal sequence complementary to a portion of the RNA, the method further comprising the steps of:
annealing the terminal sequences of bound identification vehicles with complementary portions of the RNA fragments bound to proteins to which the binding portions of the identification vehicles are themselves bound, thereby forming complexes each having a protein, a binding portion, and a combined nucleic acid strand including the oligonucleotide portion of the identification vehicle and a protein-bound RNA fragment; and
extending the oligonucleotides along the RNA fragments to which they are bound to form, from each oligonucleotide, a DNA strand including the oligonucleotide and a terminal portion complementary to at least a portion of the bound RNA fragment; and
isolating the DNA strands, wherein the determining step identifies the protein and a sequence of at least a portion of the DNA fragment.

5. The method of claim 1, wherein two or more different identification vehicles against the same proteins in the mixture are present in the incubation reaction.

6. The method of claim 1, wherein the oligonucleotide is a single strand DNA, double strand DNA, triple strand DNA, or quadruple strand DNA, or single strand RNA, or double strand RNA, triple strand RNA, or quadruple stand RNA molecule.

7. The method of claim 1, wherein the oligonucleotide portion includes a first oligonucleotide strand attached to the binding portion and a second oligonucleotide strand complementary to the first oligonucleotide strand and annealed thereto.

8. The method of claim 7, wherein the second oligonucleotide strand includes unblocked 3′- and 5′-ends.

9. The method of claim 7, wherein the step of incubating the identification vehicles with the mixture includes the binding portions of at least some of the vehicles binding to corresponding proteins in the mixture, wherein at least some of the corresponding proteins are bound to the same nucleic acid fragment; and
wherein the step of ligating the oligonucleotide portions of bound identification vehicles includes ligating to each other the oligonucleotide portions of identification vehicles bound to corresponding proteins bound to the same nucleic acid fragment, and ligating the oligonucleotide portions to the nucleic acid fragment, thereby forming complexes each having proteins, binding vehicles, and a combined nucleic acid strand including the oligonucleotides of each identification vehicle and a protein-bound nucleic acid fragment.

10. The method of claim 1, wherein the oligonucleotide portion is the binding portion of the identification vehicle.

11. The method of claim 1, wherein the oligonucleotide portion is a modified oligonucleotide.

12. The method of claim 1, wherein the modified oligonucleotide is modified by incorporation of at least one of an inhibitor of nuclease degradation, a fluorescent dye, a dark quencher, a locked nucleic acid, an unlocked nucleic acid, a modified base, a modified sugar, a threose nucleic acid, a glycol nucleic acid, a peptide nucleic acid, a zip nucleic acid, a triazole-linked deoxyribonucleic acid, a morpholino synthetic nucleic acid, a spacer, or biotin.

13. The method of claim 1, wherein the binding portion is one of an antibody, Fab antibody fragment, F(ab′)₂ antibody fragment, Fab′ antibody fragment, single-chain variable fragment, dimeric single-chain variable fragment, single domain antibody fragment, bi-specific antibody, heavy-chain antibody, oligonucleic acid aptamer, or a peptide aptamer.

14. The method of claim 1, further comprising the step of removing unbound identification vehicles from the mixture subsequent to the step of incubating the identification vehicles with the mixture.

15. The method of claim 1, further comprising the step of isolating the combined nucleic acid strands prior to the sequencing step.
